(19) United States
(12) Patent Application Publication
(10) Pub. No.: US 2002/0110604 A1
(43) Pub. Date: Aug. 15, 2002

(54) Composition exhibiting synergistic antioxidant activity

(75) Inventors: John G. Babish, Brooktondale, NY (US); Terrence Howell, Freeville, NY (US)

Correspondence Address: THORPE NORTH WESTERN, 8180 SOUTH 700 EAST, SUITE 200, P.O. BOX 1219, SANDY, UT, 84070, US

(73) Assignee: Ashni Naturaceuticals, Inc.

(21) Appl. No.: 09/925,632

(22) Filed: Aug. 9, 2001

Related U.S. Application Data
(60) Provisional application No. 60/224,678, filed on Aug. 11, 2000.

(51) Int. Cl. A61K 38/06; A61K 31/726; A61K 31/7048; A61K 31/704; A61K 31/7008; A61K 31/185; A61K 31/385

(52) U.S. Cl. 424/725; 514/18; 514/33; 514/62; 514/27; 514/54; 514/475; 514/436; 514/456; 514/763; 514/572

(57) ABSTRACT

A novel formulation is provided that serves to synergistically inhibit the generation of free radicals and oxidative stress in animals. The formulation comprises as a first component a carotenoid species, and, as a second component, at least one member selected from the group consisting of lipoic acid, dihydrolipoic acid (DHLA), a stilbene species, ergothioneine, a flavone species, a triterpene species, ascorbic acid and derivatives thereof.

### CROSS REFERENCE TO RELATED APPLICATIONS

This application claims benefit of U.S. Provisional Application No. 60/224,678 filed Aug. 11, 2000.

### FIELD OF THE INVENTION

The present invention relates generally to a composition exhibiting synergistic antioxidant activity. More particularly, the composition comprises, as a first component, a carotenoid species, and, as a second component, at least one member selected from the group consisting of lipoic acid, dihydrolipoic acid (DHLA), a stilbene species, ergothioneine, a flavone species, a triterpene species and ascorbic acid or derivatives thereof. The composition exhibits synergistic antioxidant activity.

### BACKGROUND OF THE INVENTION

Oxygen is essential for aerobic life, but is also a precursor to the formation of harmful reactive oxygen species (ROS). Oxidative stress refers to the cytotoxic consequences of a mismatch between the production of free radicals and the ability of the cell to defend against them. Oxidative stress can thus occur when the formation of ROS increases, scavenging of ROS or repair of oxy-modified macromolecules decreases, or both. ROS may be oxygen-centered radicals possessing unpaired electrons, such as superoxide and hydroxyl radicals, or covalent molecules, such as hydrogen peroxide.

Superoxide and hydrogen peroxide are relatively nonreactive toward biological molecules. Hydroxyl radicals, on the other hand, are highly reactive. Under physiological conditions, superoxide is converted to hydrogen peroxide by the enzyme superoxide dismutase (SOD) or by interaction with transition metals. Hydrogen peroxide is in turn reduced to water by glutathione peroxidase or converted to oxygen and water by catalase. Thus the hydroxyl radical represents the greatest threat to cell viability.

ROS, especially hydroxyl radicals, can produce functional alterations in lipids, proteins, and nucleic acids. The incorporation of molecular oxygen into polyunsaturated fatty acids initiates a chain reaction in which ROS, including hydroxyl radicals, hydrogen peroxide, and peroxyl and alkoxyl radicals are formed. Oxidative lipid damage, termed lipid peroxidation, produces a progressive loss of membrane fluidity, reduces membrane potential, and increases permeability to ions such as calcium. ROS can damage proteins and change amino groups on amino acids into carbonyls, resulting in the inactivation of the proteins. DNA and RNA are also targets of ROS. Hydroxyl radicals modify ribose phosphates, pyrimidine nucleotides and nucleosides and react with the sugar phosphate backbone of DNA causing breaks in the DNA strand.

Because ROS and the associated oxidative stress can produce fundamental cellular damage, primary or secondary oxidative insults have been implicated in many diseases. Table 1 below provides a list of physiological insults in which oxidative stress and ROS are believed to play a significant role and are therefore appropriate targets for normalization, prevention or treatment by antioxidants.

TABLE 1

| Physiological Insults | |
|---|---|
| Generating Oxidative Stress | Affected Tissues or Systems |
| Addison's Disease | Adrenal |
| Aging | Skin and other systems |
| Allergies | Inflammatory cells |
| Alzheimer Disease | Nerve cells |
| Angioplasty | Arterial epithelial cells |
| Arthritis | Inflammatory cells |
| Asthma | Immune cells |
| Atherosclerosis | Vessel wall |
| Cigarette Smoking | Lung, mouth, throat and blood vessels |
| Colon Cancer | Intestine |
| Chacaxia | Muscular and Nervous |
| Crohn's Disease | Intestine |
| Cystic Fibrosis | Lungs |
| Diabetes (type I and type II) | Pancreas and various systems |
| Eczema | Skin/Inflammatory cells |
| Graves' Disease | Thyroid |
| Guillain-Barre Syndrome | Nerve cells |
| Head Injury | Brain |
| Hemodialysis | Kidney |
| Hepatitis | Liver |
| HIV-1 Infection | Muscular and Immune systems |
| Hyperlipidemia | Liver and Arterial vessels |
| Hypothyroidism | Thyroid |
| Inflammation | Immune cells |
| Inflammatory Bowel Disease | Intestine |
| Leukemia | Immune cells |
| Lymphomas | Immune cells |
| Multiple Sclerosis | Nerve cells |
| Myasthenia Gravis | Neuromuscular junction |
| Nuclear Factor kappaB | Immune cells |
| Activation | |
| Neurodegeneration | Central nervous system |
| Physical Exertion | Muscular and Immune systems |
| Psoriasis | Skin |
| Primary Billiary Cirrhosis | Liver |
| Reperfusion Injury | Head and Heart |
| Rheumatoid Arthritis | Joint lining |
| Solid Tumors | Various |
| Systemic Lupus Erythematosis | Multiple tissues |
| Tumor Necrosis Factor-alpha | Various Systems |
| Expression | |
| Uveitis | Eye |

Numerous epidemiological investigations have suggested that consumption of antioxidants in the form of fresh fruits and vegetables provides protection from cancer, cardiovascular disease, autoimmune disease and neurodegeneration. Furthermore, in vitro studies support the palliative effects of single, purified antioxidant treatment in a variety of model systems. In particular, carotenoids have been a focus of study with respect to decreasing oxidative stress as well as cancer prevention and intervention.

Carotenoids (FIG. 1[A]) are a family of over 700 natural, lipid-soluble pigments that are only produced by phytoplankton, algae, plants and a limited number of fungi and bacteria. The carotenoids are responsible for the wide variety of colors they provide in nature, most conspicuously in the yellow and red colors of fruits and leaves. In plants and algae, carotenoids along with chlorophyll and other light-harvesting pigments are vital participants in the photosynthetic process.

Biologically, carotenoids are distinguished by their capacity to interact with singlet oxygen and free radicals. Among the carotenoids, a growing body of scientific literature describes astaxanthin as one of the best antioxidants. Due to its unique molecular structure among carotenoids (a carbonyl and hydroxy group on each of the terminal aromatic rings), astaxanthin has both a potent quenching effect against singlet state oxygen and a powerful scavenging ability for free radicals. Thus, astaxanthin serves as an extremely effective antioxidant against these reactive species. However, experience with intervention trials has shown that supplementation with a single antioxidant may produce untoward, stimulatory effects on cancer growth.

Numerous epidemiological investigations have shown that cancer risk is inversely related to the consumption of green and yellow vegetables and fruits. Since beta-carotene is present in abundance in these vegetables and fruits, it has been investigated extensively as a possible cancer-preventive agent. However, various other carotenoids also have anti-carcinogenic activity. For example, lutein, zeaxanthin, lycopene, phytoene, fucoxanthin, peridinin and astaxanthin seem to be promising. Among these later carotenoids, astaxanthin has most recently demonstrated the greatest antioxidant activity.

However, as a result of clinical studies, the role of carotenoids as anticancer supplements has recently been questioned. For example, the incidence of non-melanoma skin cancer was unchanged in patients receiving a β-carotene supplement A recent study also shows that smokers gained no benefit from supplemental β-carotene with respect to lung cancer incidence and possibly even suffered a deleterious effect. This inference also extends to numerous other diseases associated with oxidative stress such as Alzheimer's disease, diabetes and cardiovascular disease.

Consequently, it has been concluded that it is necessary to identify combinations of two or more antioxidants that would work together synergistically in order to have a reasonable probability of clinical effectiveness for cancer prevention or intervention. It would be useful to produce a potent combination of antioxidants that function synergistically to inhibit the generation of free radicals.

Lipoic acid (FIG. 2[A]), which plays an essential role in mitochondrial dehydrogenase reactions, has recently gained considerable attention as an antioxidant. Exogenous alpha-lipoic acid is reduced intracellularly by at least two and possibly three enzymes, and through the actions of its reduced form, it influences a number of cell processes. These include direct radical scavenging, recycling of other antioxidants, accelerating GSH synthesis, and modulating transcription factor activity, especially that of NF-kappa B. Lipoate, or its reduced form, dihydrolipoate (FIG. 2[B]), reacts with reactive oxygen species such as superoxide radicals, hydroxyl radicals, hypochlorous acid, peroxyl radicals, and singlet oxygen. It also protects membranes by interacting with vitamin C and glutathione, which may in turn recycle vitamin E. In addition to its antioxidant activities, dihydrolipoate may exert prooxidant actions through reduction of iron.

The administration of alpha-lipoic acid has been shown to be beneficial in a number of oxidative stress models such as ischemia-reperfusion injury, diabetes (both alpha-lipoic acid and dihydrolipoic acid (DHLA) exhibit hydrophobic binding to proteins such as albumin, which can prevent glycation reactions), cataract formation, HIV activation, neurodegeneration and radiation injury. Furthermore, lipoate can function as a redox regulator of proteins such as myoglobin, prolactin, thioredoxin and NF-kappa B transcription factor.

As scavengers of ROS, lipoic acid and DHLA, display antioxidant activity in most experiments, whereas, in particular cases, pro-oxidant activity has been observed. DHLA has the capacity to regenerate the endogenous antioxidants vitamin E, vitamin C and glutathione. Through the lipoamide dehydrogenase-dependent reduction of lipoic acid, the cell can draw on its NADH pool for antioxidant activity and additionally on its NADPH pool, which is usually consumed during oxidative stress. Within drug-related antioxidant pharmacology, lipoic acid is a model compound that enhances understanding of the mode of action of antioxidants in drug therapy.

Resveratrol is a species of the stilbene genus (FIG. 3[A]) which are natural compounds occurring in a number of plant families including Vitaceae. Included within this family is Vitis vinifera L., which is the most important species grown worldwide for grape and wine production. Resveratrol (3, 4′, 5-trihydroxystilbene, FIG. 3[B]) is a major stilbene produced by grapevines. Given that it is present in grape berry skins but not in flesh, white wine contains very small amounts of resveratrol, compared to red wine. The concentrations in the form of trans- and cis- isomers of aglycone and glucosides are subjected to many variables. In red wine, concentrations of the trans-isomer, which is the major form, generally range between 0.1 and 15 mg/L. As a phenolic compound, resveratrol contributes to the antioxidant potential of red wine and thereby may play a role in the prevention of human cardiovascular diseases. Resveratrol has been shown to modulate the metabolism of lipids, inhibit the oxidation of low-density lipoproteins and the aggregation of platelets. Moreover, as a phytoestrogen, resveratrol may provide cardiovascular protection. This compound also possesses anti-inflammatory and anticancer properties.

Ergothioneine—L-Ergothioneine or 2-mercapto-Na,Na,Na-trimethyl-L-histidine (FIG. 4) is a natural molecule that was isolated from the rye ergot fungus Claviceps purpurea. It was subsequently identified in rat erythrocytes and liver and then in numerous other animal and human tissues. It is biosynthesized exclusively by fungi and mycobacteria. In plants the roots assimilate ergothioneine, after fungal synthesis inside the conidia. In humans, it is assimilated solely through food. Ergothioneine, by virtue of its antioxidant properties, can be used as a food additive, dietary supplement, medicine or in cosmetics.

Genistein (FIG. 5[B]), a flavone (FIG. 5[A]) found in soy, has been reported to have weak estrogenic and antiestrogenic properties, to be an antioxidant, to inhibit toposiomerase II and angiogenesis, and to induce cell differentiation. Epidemiological evidence supports a role of genistein and soy protein in the prevention of both breast and prostate cancer. Mechanistically, in vitro data support the role of genistein as a tyrosine kinase inhibitor or antioxidant. No synergies of compounds interacting with genistein have been reported to date.

Members of the triterpene genus (FIG. 6[A]), such as represented by the oleanolic acid species (FIG. 6[B]), are commonly found in plants and are useful for their antioxidant properties. The antioxidant effects of these compounds have been described in the literature since 1960. Oleanolic acid is capable of inhibiting the generation of reactive oxygen intermediates and restoring tissue glutathione levels following stress.

Ascorbic acid (Vitamin C) (FIG. 7) is the most abundant water-soluble antioxidant in the body and in plants, exhibiting antioxidant activity primarily in extracellular fluid. Its actions are most notable in combating the free radicals of polluted air and cigarette smoke. Not only does ascorbic acid scavenge many free radicals, but it helps return vitamin E to its active form. Statistical description of synergistic interactions of vitamin C with beta-carotene or other carotenoids have not been previously described.

It would be useful to provide compounds that would function synergistically with a carotenoid species, such as astaxanthin, to increase the antioxidant activity of the carotenoid species.

### SUMMARY OF THE INVENTION

The present invention provides a composition having a synergistic inhibitory effect on biological oxidative processes involving free radicals or singlet oxygen. The present invention provides a composition comprising, as a first component, a carotenoid species, and, as a second component, at least one member selected from the group consisting of lipoic acid, dihydrolipoic acid (DHLA), a stilbene species, ergothioneine, a flavone species, a triterpene species, ascorbic acid and derivatives thereof. The composition exhibits synergistic antioxidant activity.

The composition of the present invention must contain, at a minimum, two species one each representing the first component (a carotenoid) and a second component selected from the group consisting of lipoic acid, dihydrolipoic acid (DHLA), a stilbene species, ergothioneine, a flavone species, a triterpene species, ascorbic acid and derivatives thereof. However, additional species or mixtures of species within the various genera may be present in the composition which is limited in scope only by the combinations of species within the various genera that exhibit the claimed synergistic functionality.

Preferably, the carotenoid species is a member selected from the group consisting of astaxanthin, beta-carotene, lutein, lycopene, zeaxanthn, and cantaxanthin. More preferably, the carotenoid species is a member selected from the group consisting of astaxanthin, beta-carotene, lutein, and lycopene . The most preferred cartenoid species is astaxanthin.

Preferably, the triterpene species is a member be selected from the group consisting of oleanolic acid, ursolic acid, betulin, tripterin, and glycyrrhizic acid. More preferably, the triterpene species is a member selected from the group consisting of oleanolic acid and ursolic acid. The most preferred triterpene species is oleanolic acid.

Preferably, the stilbene species is a tran-stilbene selected from the group consisting of resveratrol and piceatannol. More preferably, the stilbene species is resveratrol.

Preferably, the flavone species is a member selected from the group consisting of genistein, daidzein, glycitein, formonoetin, genistein, and daizin. More preferably, the flvone species is a member selected from the group consisting of genistein, daidzein, glycitein. The most preferred flavone species is genistein.

One specific embodiment of the present invention is a composition formulation comprising an effective amount of astaxanthin and at least one compound selected from the group consisting of lipoic acid, resveratrol, ergothioneine, genistein, oleanolic acid, or ascorbic acid. The composition functions synergistically to inhibit the generation of free radicals and oxidative stress.

The present invention further provides a composition of matter which enhances the normal functioning of the body in times of oxidative stress resulting from a chronic debilitating disease.

The present invention further provides a method of dietary supplementation and a method of treating oxidative stress or oxidative stress-based diseases in a warm-blooded animals which comprises providing to the animal suffering symptoms of oxidative stress the composition of the present invention containing a second component which specifically and synergistically enhances the antioxidant activity of astaxanthin and continuing to administer such a dietary supplementation of the composition until said symptoms are eliminated or reduced.

### BRIEF DESCRIPTION OF THE DRAWINGS

FIGS. 1[A] and [B] respectively, illustrates the general chemical structure of the carotenoid genus and astaxanthin (3, 3′-dihydroxy-β,β-carotene-4, 4′-dieto-β-carotene) as a species within that genus

FIGS. 2[A] and [B] respectively, illustrates the chemical structures of alpha-lipoic acid (1,2-dithiolane-3-pentanoic acid) and dihydrolipoic acid (DHLA).

FIGS. 3 [A] and [B] respectively, illustrates the general chemical structure of the trans-stilbene genus and resveratrol (3, 4′, 5-trihydroxystilbene) as a species within that genus.

FIG. 4 illustrates the chemical structure of L-ergothioneine (2-mercapto-Na,Na,Na-trimethyl-L-histidine).

FIGS. 5 [A] and [B] respectively, illustrates the general chemical structure of the flavone genus and genistein (4′, 5, 7-trihydroxyisooflavone) as a species within that genus.

FIGS. 6 [A] and [B] respectively, illustrates the general chemical structure of the triterpene genus and oleanolic acid (3β-3-hydroxyolean-12-en-28-oic acid) as a species within that genus.

FIG. 7 illustrates the chemical structure of ascorbic acid (L-ketothreohexuronic acid).

FIG. 8 illustrates relative antioxidant activity of ginseng alone and the combination of ginseng and algal meal in a weight ratio of 9:1.

FIG. 9 illustrates relative antioxidant activity of garlic alone and the combination of garlic and algal meal in a weight ratio of 24:1.

FIG. 10 illustrates relative antioxidant activity of ginkgo alone and the combination of ginkgo and algal meal in a weight ratio of 19:1.

### DETAILED DESCRIPTION OF THE INVENTION

Before the present composition and methods of making and using thereof are disclosed and described, it is to be understood that this invention is not limited to the particular configurations, as the process steps and materials may vary somewhat. It is also intended to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise.

The present invention provides a composition having synergistic antioxidant activity. More particularly, the composition comprises, as a first component, a carotenoid species, and, as a second component, at least one member selected from the group consisting of lipoic acid, dihydrolipoic acid (DHLA), a stilbene species, ergothioneine, a flavone species, a triterpene species, ascorbic acid and derivatives thereof. Preferably, the molar ratio of the active first component, i.e. the carotenoid species, to the second component, i.e. a member selected from the group consisting of lipoic acid, dihydrolipoic acid (DHLA), a stilbene species, ergothioneine, a flavone species, a triterpene species, ascorbic acid and derivatives thereof is within a range of 50:1 to 1:100. When the second component is ascorbic acid, the molar ratio of the first and second component is within a range of 50:1 to 1:10,000. The composition provided by the present invention can be formulated as a dietary supplement or therapeutic composition. The composition functions synergistically to inhibit biological oxidation involving free radicals or singlet oxygen. Such combinations are useful as dietary supplements or as therapeutics for the physiological insults listed in Table 1.

As used herein, the term “dietary supplement” refers to compositions consumed to affect structural or functional changes in physiology. The term “therapeutic composition” refers to any compounds administered to treat or prevent a disease.

As used herein, the term “antioxidant activity” refers to an inhibitory effect on biological oxidative processes involving free radicals or singlet oxygen.

As used herein, carotenoid species, lipoic acid, dihydrolipoic acid (DHLA), a stilbene species, ergothioneine, a flavone species, a triterpene species, ascorbic acid and derivatives thereof are meant to include naturally occurring or synthetic derivatives of species within the scope of the respective genera. Natural derivatives may be obtained from common microbiological or plant sources and may exist as conjugates.

“Conjugates” of carotenoid species, lipoic acid, dihydrolipoic acid (DHLA), a stilbene species, ergothioneine, a flavone species, a triterpene species, ascorbic acid or derivatives thereof means carotenoid species, lipoic acid, resveratrol, ergothioneine, genistein, oleanolic acide, ascorbic acid or derivatives thereof covalently bound or conjugated to a member selected from the group consisting of mono- or di- saccharides, amino acids, fatty acids, sulfates, succinate, acetate and glutathione. Preferably, the fatty acid is a C₆ to C₂₂ fatty acid. Preferably, the mono- or di- saccharide is a member selected from the group consisting of glucose, mannose, ribose, galactose, rhamnose, arabinose, maltose and fructose.

Therefore, one preferred embodiment of the present invention is a composition comprising a combination of an effective amount of astaxanthin as a first component, and, as a second component, at least one member selected from the group consisting of lipoic acid, resveratrol, ergothioneine, genistein, oleanolic acid and ascorbic acid or derivatives thereof. The resulting formulation of these combinations exhibits synergistic antioxidant activity.

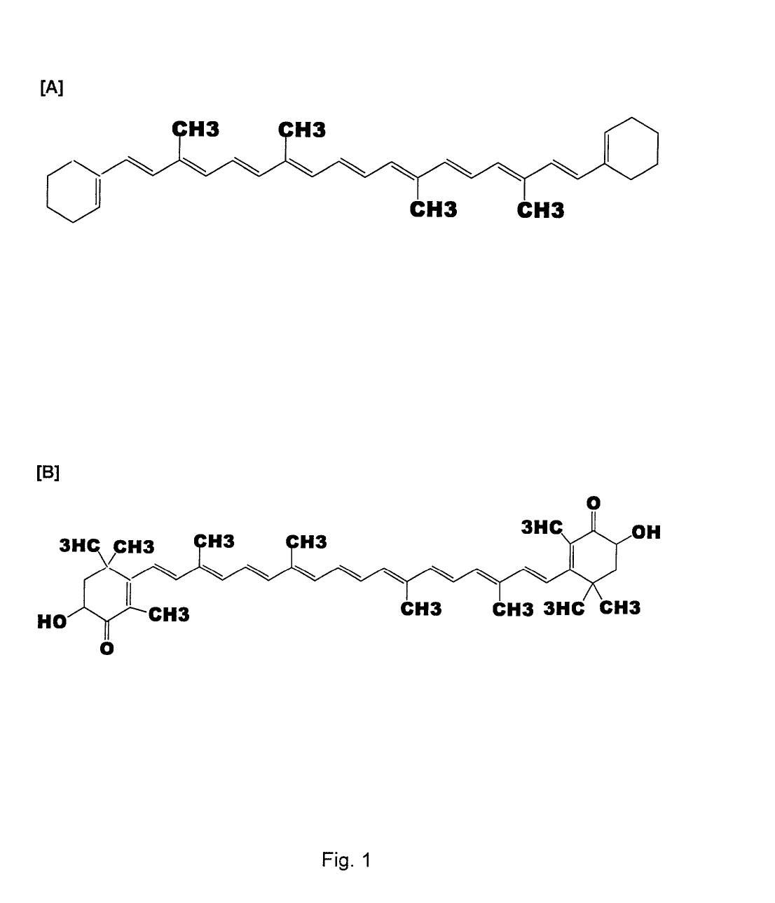

Preferably, the carotenoid or astaxanthin (FIGS. 1 [A1] and [B]) employed in the present invention is a pharmaceutical grade preparation such as can be obtained commercially, for example, from AstaCarotene AB, Gustravsberg, Sweden. The pharmaceutical grade extract must pass extensive safety and efficacy procedures. Pharmaceutical grade astaxanthin is standardized to have greater than 2 weight percent of astaxanthin and can be readily obtained from the green algae Haematococcus pluvialis. As employed in the practice of the invention, the astaxanthin extract has an astaxanthin content of about 1.0 to 95 percent by weight. Preferably, the minimum astaxanthin content is about 2 percent by weight.. Alternatively, the astaxanthin may be synthesized using standard techniques known in chemical synthesis.

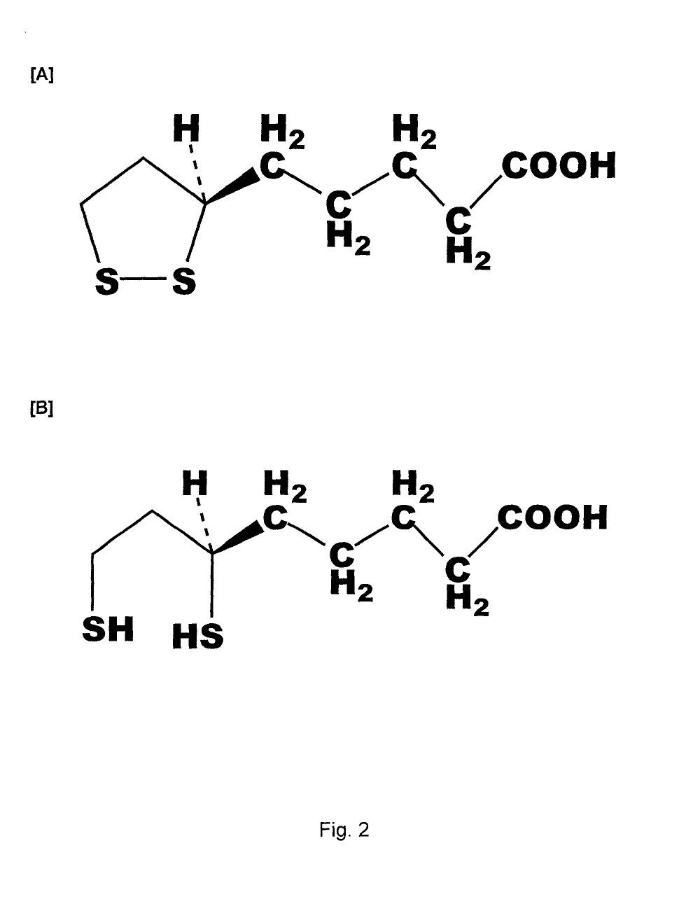

The lipoic acid or DHLA, as represented by FIGS. 2 [A] and [B] respectively, is preferably a pharmaceutical grade preparation such as can be obtained commercially, for example, as a prepartion with a pruity of greater than 95 percent from Technical Sourcing Intemations (Missoula, Mont.).

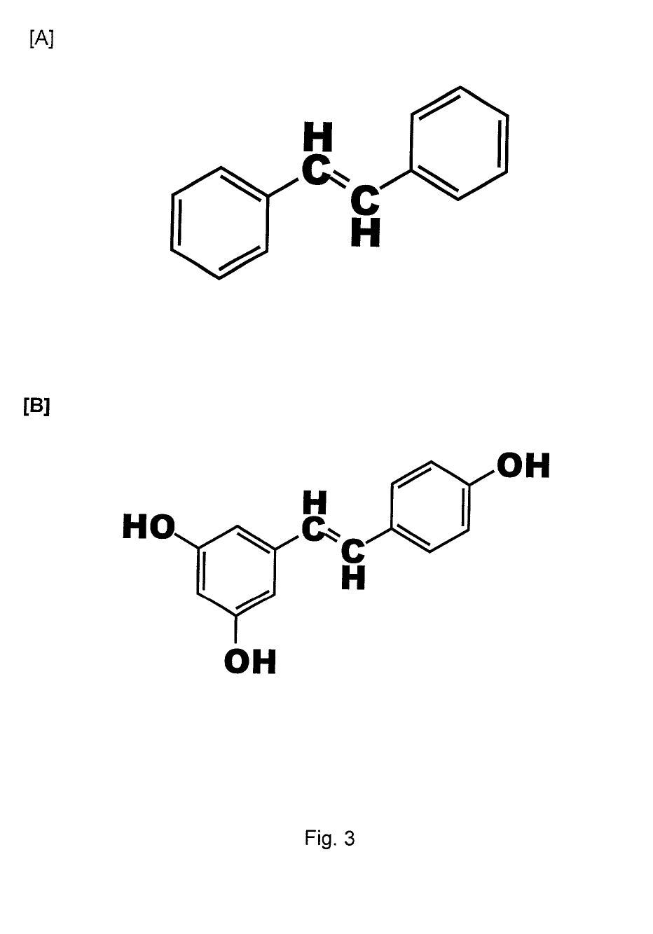

The preferred trans-stilbene resveratrol employed (FIG. 3[B]) is a pharmaceutical grade preparation that can be obtained from DNP International, Terre Haute, Ind. In general resveratrol is obtained in the form of standardized extracts of grape skins or leaves, (Vitis vinifera), White Mulberry (Morus alba), or Japenese Knotweed (Polygonum cuspidatum). Pharmaceutical grade resveratrol is equal to or greater than 5 percent by weight of resveratrol. As employed in the practice of this invention the extract has a minimum resveratrol content of 1 to 40 percent by weight.

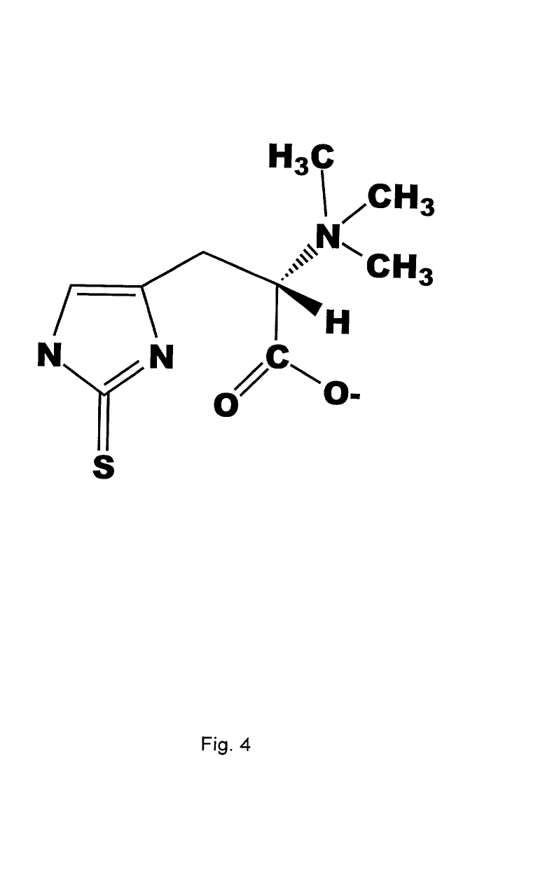

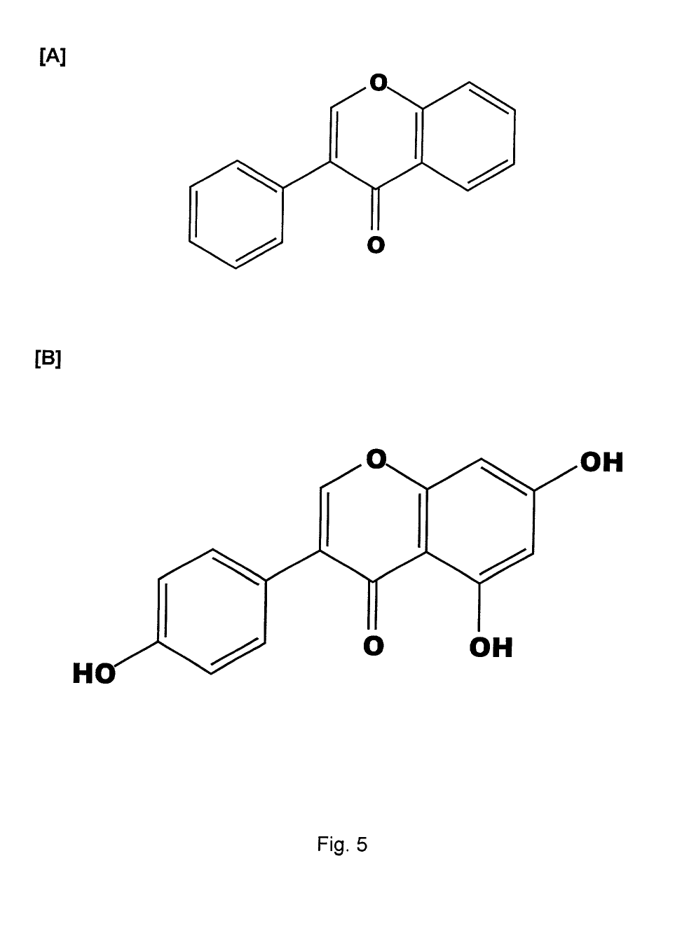

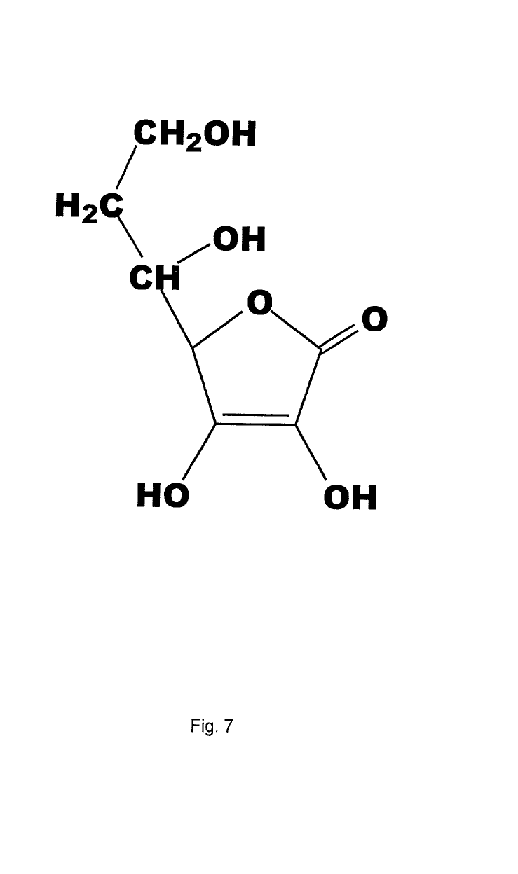

Flavones or genistein, as represented by FIGS. 5 [A] and [B] respectively, can be obtained from a product derived from Glycine max (L. MERR), Genista tinctoria, Prunus cerasus L., or Ulex europaeus L. The ergothionine (FIG. 4) can be obtained from a product derived from the Shiitake or Oyster mushrooms. The ascorbic acid (FIG. 7) can be obtained from a product derived from Malpighia glabra L., Capsicum frutescens L., or Rosa spp.

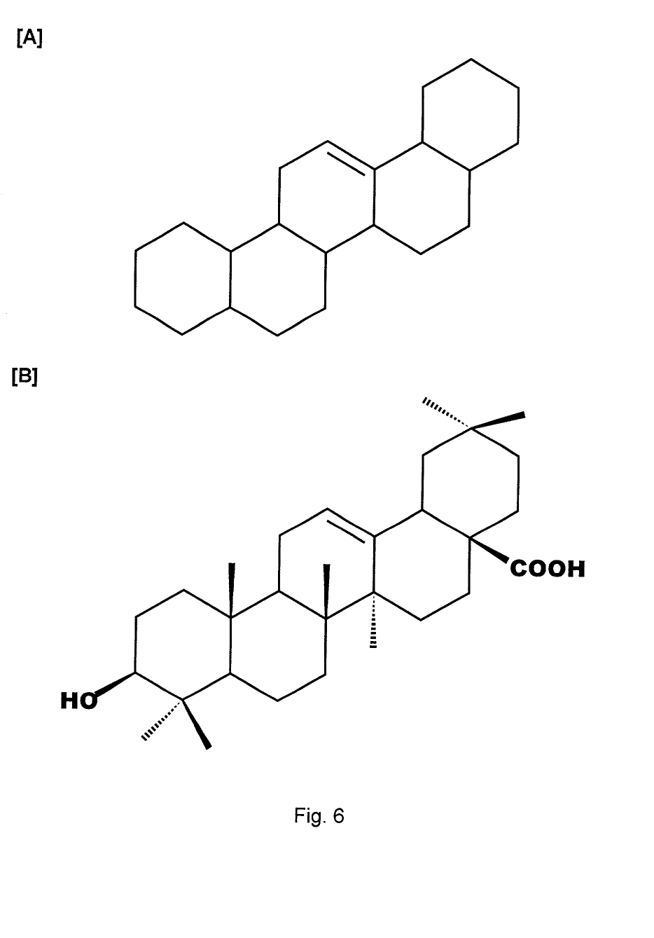

Tripterpenes as represented by FIG. 6[A], such as ursolic acid or oleanolic acid [FIG. 6[B]), are both found in a wide variety of botanicals. For example, ursolic acid can be sourced from Adina piluifera, Agrimonia eupatoria, Arbutus unedo, Arctostaphylos uva-ursi, Artocarpus heterophyllus, Catalpa bignoniodes, Catharanthus roseus, Chimaphila umbellata, Cornus florida, Cornus officinalis, Crataegus cuneata, Crataegus laevigata, Crataegus pinnatifida, Cryptostegia grandifolia, Elaeagnus pungens, Eriobotrya japonica, Eucalyptus citriodora, Forsythia suspensa, Gaultheria fragrantissima, Glechoma hederacea, Hedyotis diffusa, Helichrysum angustifolium, Humulus lupulus, Hyssopus officinalis, Ilex paraguariensis, Lavandula angustifolia, Lavandula latifolia, Leonurus cardiaca, Ligustrum japonicum, Limonia acidissima, Lycopus europeus, Malus domestica, Marubium vulgare, Melaleuca leucadendra, Melissa officinalis, Mentha spicata, Mentha x rotundifolia, Monarda didyma, Nerium oleander, Ocimum basilicum, Ocimum basilicum, Ocimum basilicum, Ocimum baslicum, Ocimum canum, Origanum majorana, Origanum vulgare, Plantago asiatica, Plantago major, Plectranthus amboinicus, Prunell vulgaris, Prunella vulgaris, Prunus cerasus, Prunus laurocerasus, Prunus persica, Prunus serotina spp serotina, Psidium guajava, Punica granatum, Pyrus communis, Rhododendron dauricum, Rhododendron ferrugineum, Rhododendron ponticum, Rosmarinus officinalis, Rubus fruticosus, Salvia officinalis, Salvia sclarea, Salvia triloba, Sambucus nigra, Sanguisorba officinalis, Satureja hortensis, Satureja montana, Sorbus aucubaria, Syringa vulgaris, Teucrium chamaedrys Teucrium polium, Teucrium spp, Thevetia peruviana, Thymus serpyllum, Thymus vulgaris, Uncaria tomentosa, Vaccinium corymobosum, Vaccinium myrtillus, Vaccinium vitis idaea, Verbena officinalis, Viburnum opulus var. opulus, Viburnum prunifolium, Vinca minor or Zizyphus jujuba.

Similarly, oleanolic acid is found in Achyranthes aspera, Achyranthes bidentiata, Adina piluifera, Ajpocynum cannabinum, Akebia quinata, Allium cepa, Allium sativum, Arctostaphylos uva-ursi, Calendula officinalis, Catharanthus roseus, Centaurium erythraea, Chenopodium album, Citrullus colocynthis, Cnicus benedictus, Cornus officinalis, Crataegus pinnatifida Cyperus rotundus, Daemonorops draco, Diospyros kaki, Elaeagnus pungens, Eleutherococcus senticosus, Eriobotrya japonica, Eugenia caryophyllata, Forsythia suspensa, Glechoma hederacea, Harpagophtum procumbens, Hedera helix, Hedyotis diffusa, Helianthus annuus, Hemsleys amabilis, Humulus lupulus, Hyssopus officinalis, Ilex rotunda, Lavandula latifolia, Leonurus cardiaca, Ligustrum japonicum, Ligustrum lucidum, Liquidambar orientalis, Liquidambar styraciflua, Loranthus parasiticus, Luffa aegyptiaca, Melaleuca leucadendra, Melissa officinalis, Mentha spicata, Mentha x rotundifolia, Momordica cochinchinensis, Myristica fragrans, Myroxylon balsamum, Nerium oleander, Ocimum suave, Ociumum basilicum, Olea europaea, Origanum majorana, Origanum vulgare, Paederia scandens, Panax ginseng, Panax japonicus, Panax quinquefolius, Patrinia scabiosaefolia, Phytolacca americana, Plantago major, Plectranthus amboinicus, Prunella vulgaris, Prunus cerasus, Psidium guajava, Pulsatilla chinenisis, Quisqualis indica, Rosmarinus officinalis, Salvaia officinalis, Salvia sclarea, Salvia triloba, Sambucus nigra, Satureja hortensis, Satureja montana, Swertia chinensis, Swertia diluta, Swertia mileensis, Syzygium aromaticum, Thymus serpyllum, Thymus vulgaris, Trachycarpus fortunei, Uncaria tomentosa, Vaccinium corymbosum, Vaccinium myrtillus, Viburnum prunifolium, Viscum album, Vitis vinifera, and Zizyphus jujuba.

The preferred botanical sources for ursolic acid is a member selected from the group consisting of Ligustrum japonicum, Plantago asiatica, Plantago major, Prunus species, Uncaria tomentosa, Zizyphus jujuba, Cornus officinalis, Eucalyptus citriodora, Forsythia suspensa, Lavandula latifolia, Malus domestica, Nerium oleander, Ocimum baslicum, Punica granatum, Pyrus communis, Rosmarinus officinalis, Salvia triloba, Sorbus aucubaria, Vaccinium myrtillus, Vaccinium vitis-idaea, and Viburnum opulus var. opulus. The most preferred botanical source for ursolic acid is a member selected from the group consisting of Ligustrum japonicum, Plantago asiatica, Plantago major, Prunus species, Uncaria tomentosa, and Zizyphus jujuba.

The preferred botanical sources for oleanolic acid is a member selected from the group consisting of Eleutherococcus senticosus, Ligustrum japonicum, Ligustrum lucidum, Panax ginseng, Panax japonicus, Panax quinquefolius, Plantago major, Vitis vinifera, Zizyphus jujuba, Achyranthes bidentiata, Allium cepa, Allium sativum, Cornus officinalis, Daemonorops draco, Forsythia suspensa, Prunus cerasus, Quisqualis indica, Rosmarinus officinalis, Salvia triloba, Syzygium aromaticum, Thymus vulgaris, Uncaria tomentosa, Vaccinium corymbosum, and Vaccinium myrtillus. The most preferred botanical source for oleanolic acid is a member selected from the group consisting of Eleutherococcus senticosus, Ligustrum japonicum, Ligustrum lucidum, Panax ginseng, Panax japonicus, Panax quinquefolius, Plantago major, Vitis vinifera and Zizyphus jujuba.

Without limiting the invention, the action of the second component of the composition is thought to provide a dual, synergistic, antioxidant effect with the first component. The second compound can also provide hepatoprotection, antitumor promotion, antihyperlipidemia, antihyperglycemia, and protection against ulcer formation from oxidative stress.

A daily dose (mg/day) of the present dietary supplement would be formulated to deliver: 1 to 50 mg of a carotenoid species, and 10 to 1200 mg lipoic acid or dihydrolipoic acid, 1 to 1000 mg of a stilbene species, 1 to 50 mg of ergothioneine, 0.5 to 500 mg of a flavone species, 2 to 1000 mg of a triterpene species, or 50 to 10,000 mg ascorbic acid. Preferably, the daily dose (mg/day) of the present dietary supplement would be formulated to deliver: 3 to 15 mg of a cartenoid species, and 100 to 600 mg lipoic acid or dihydrolipoic acid, 5 to 40 mg of a stilbene species, 3 to 20 mg of ergothioneine, 5 to 50 mg of a flavone species, 25 to 150 mg of a triterpene species, or 500 to 2000 mg of ascorbic acid.

The composition of the present invention for topical application would contain 0.001 to 10 wt %, preferably 0.05 to 2 wt %, of the first component of a carotenoid species, and, 0.001 to 10 wt %, preferably 0.05 to 2 wt %, of the second component selected from the group consisting of lipoic acid, dihydrolipoic acid (DHLA), a stilbene species, ergothioneine, a flavone species, a triterpene species, ascorbic acid and derivatives thereof.

The preferred composition of the present invention would produce serum or tissue concentrations in the following range: 0.01 to 5,500 μM of a cartenoid species, and 0.08 to 50 μM lipoic acid or dihydrolipoic acid, 0.005 to 50 μM of a stilbene species, 0.01 to 3,000 μM of ergothioneine, 0.02 to 800 μM of a flavone species, 0.05 to 3,500 μM of a triterpene species, or 0.01 to 500 μM ascorbic acid

In addition to the combination of active ingredients selected from the group consisting of a carotenoid species, lipoic acid, dihydrolipoic acid (DHLA), a stilbene species, ergothioneine, a flavone species, a triterpene species, ascorbic acid and derivatives thereof, the present composition for dietary application may include various additives such as other natural components of intermediary metabolism, vitamins and minerals, as well as inert ingredients such as talc and magnesium stearate that are standard excipients in the manufacture of tablets and capsules.

As used herein, “pharmaceutically acceptable carrier” includes any and all solvents, dispersion media, coatings, isotonic and absorption delaying agents, sweeteners and the like. These pharmaceutically acceptable carriers may be prepared from a wide range of materials including, but not limited to, diluents, binders and adhesives, lubricants, disintegrants, coloring agents, bulking agents, flavoring agents, sweetening agents and miscellaneous materials such as buffers and absorbents that may be needed in order to prepare a particular therapeutic composition. The use of such media and agents for pharmaceutically active substances is well known in the art. Except insofar as any conventional media or agent is incompatible with the active ingredients, its use in the present composition is contemplated. In one embodiment, talc and magnesium stearate are included in the present formulation. When these components are added they are preferably, the Astac Brand 400 USP talc powder and the veritable grade of magnesium stearate. Other ingredients known to affect the manufacture of this composition as a dietary bar or functional food can include flavorings, sugars, amino-sugars, proteins and/or modified starches, as well as fats and oils.

The dietary supplements, lotions or therapeutic compositions of the present invention can be formulated in any manner known by one of skill in the art. In one embodiment, the composition is formulated into a capsule or tablet using techniques available to one of skill in the art. In capsule or tablet form, the recommended daily dose for an adult human or animal would preferably be contained in one to six capsules or tablets. However, the present compositions may also be formulated in other convenient forms such as, an injectable solution or suspension, a spray solution or suspension, a lotion, gum, lozenge, food or snack item. Food, snack, gum or lozenge items can include any ingestable ingredient, including sweeteners, flavorings, oils, starches, proteins, fruits or fruit extracts, vegetables or vegetable extracts, grains, animal fats or proteins. Thus, the present composition can be formulated into cereals, snack items such as chips, bars, gum drops, chewable candies or slowly dissolving lozenges.

The present invention contemplates treatment of all types of oxidative stress-based diseases, both acute and chronic. The present formulation reduces the symptoms of oxidative stress and thereby promotes healing of, or prevents further damage to, the affected tissue. A pharmaceutically acceptable carrier may also be used in the present compositions and formulations.

According to the present invention, the animal may be a member selected from the group consisting of humans, non-human primates, such as dogs, cats, birds, horses, ruminants or other warm blooded animals. The invention is directed primarily to the treatment of human beings. Administration can be by any method available to the skilled artisan, for example, by oral, topical, transdermal, transmucosal, or parenteral routes.

The following examples are intended to illustrate but not in any way limit the invention.

### EXAMPLE 1

### Antioxidant Synergy of Astaxanthin and Lipoic Acid

This example illustrates the antioxidant synergy between astaxanthin and lipoic acid.

The total antioxidant capacity was measured by the Total Oxyradical Scavenging Capacity (TOSC) Assay. The TOSC assay is based on the reaction between peroxyl radicals (or hydroxyl or alkoxyl radicals, which are generated by thermal homolysis of 2,2′-azobis-amidinopropane, ABAP) and α-keto-γ-methiolbutyric acid (KMBA), which is oxidized to ethylene on reaction with various reactive oxygen species. Peroxidation, as measured as ethylene production by gas chromatography using a flame ionization detector, is suppressed in a dose-response manner for antioxidants or phytochemicals.

The area under the kinetic curve was calculated by integration. The TOSC value of each concentration was then quantified according to the following equation:

TOSC=100−[(ψSA/ψCA)×100]

where ψSA and ψCA were the integrated area from the curve defining the sample and control reactions, respectively. The control contains all reagents except the test material. Samples with positive TOSC values are designated antioxidant, while those with negative TOSC values are designated prooxidant.

Synergy between astaxanthin and lipoic acid was assessed using CalcuSyn (BIOSOFT, biosoft.com). This statistical package performs multiple drug dose-effect calculations using the Median Effect methods described by T-C Chou and P. Talaly (Trends Pharmacol. Sci. 4:450-454), hereby incorporated by reference.

Briefly, it correlates the “Dose” and the “Effect” in the simplest possible form: fa/fu=(C/Cm)^{m}, where C is the concentration or dose of the compound and Cm is the median-effective dose signifying the potency. Cm is determined from the x-intercept of the median-effect plot. The fraction affected by the concentration of the test material is fa and the fraction unaffected by the concentration is fu (fu=1−fa). The exponent m is the parameter signifying the sigmoidicity or shape of the dose-effect curve. It is estimated by the slope of the median-effect plot.

The median-effect plot is a plot of x=log (C) vs y=log (fa/fu) and is based on the logarithmic form of Chou's median-effect equation. The goodness of fit for the data to the median-effect equation is represented by the linear correlation coefficient r of the median-effect plot. Usually, experimental data from enzyme or receptor systems have an r>0.96, from tissue cultures an r>0.90 and from animal systems an r>0.85.

Synergy of test components is quantified using the combination index (CI) parameter. The combination index of Chou-Talaly is based on the multiple drug-effect and is derived from enzyme kinetic models (Chou, T.-C. and Talalay, P. (1977) A simple generalized equation for the analysis of multiple inhibitions of Michaelis-Menten kinetic systems. J. Biol. Chem. 252:6438-6442). The equation determines only the additive effect rather than synergism or antagonism. However, synergism is defined as a more than expected additive effect, and antagonism as a less than expected additive effect as proposed by Cho and Talalay in 1983 (Trends Pharmacol. Sci. (1983) 4:450-454). Using the designation of CI=1 as the additive effect, there is obtained for mutually exclusive compounds that have the same mode of action or for mutually non-exclusive drugs that have totally independent modes of action the following relationships: CI<1, =1, and >1 indicating synergism, additivity and antagonism, respectively.

Table 2 indicates the EC50 value of 6.07 μM of the combination of astaxanthin with lipoic acid when the components exist in a ratio of approximately 17:1 (astaxanthin: lipoic acid).

TABLE 2
Statistical results of combining astaxanthin with lipoic acid

| Combination | EC50 (μM) | COMBINATION INDEX* |
|---|---|---|
| Astaxanthin: Lipoic Acid | 6.07 | 0.675 |
| (17:1) | | |

*Exhibited significant (p <0.5) synergy with CI <1.0.

This combination effectively increased the antioxidant potency of astaxanthin 106-fold; astaxanthin alone exhibited an EC50 of 642 μM. In this experiment, it was discovered that a combination of 17 parts of astaxanthin to 1 part of lipoic acid provided a statistically and biologically significant increase in antioxidant efficacy of astaxanthin.

### EXAMPLE 2

### Antioxidant Synergy of Astaxanthin and Resveratrol

This example illustrates the antioxidant effect of combinations of astaxanthin and resveratrol. The experiment was performed as described in EXAMPLE 1, except that the second compound was resveratrol, obtained from Sigma (St. Louis, Mo.). Table 3 indicates an EC50 value of 3.09 μM of the combination of astaxanthin with resveratrol when the components exist in a ratio of approximately 37:1 (astaxanthin:resveratrol).

TABLE 3
Statistical results of combining astaxanthin with resveratrol

| Combination | EC50 (μM) | COMBINATION INDEX* |
|---|---|---|
| Astaxanthin: Resveratrol | 3.09 | 0.757 |
| (37:1) | | |

*Exhibited significant (p <0.5) synergy with CI <1.0.

This combination effectively increased the antioxidant potency of astaxanthin 208-fold; astaxanthin alone exhibited an EC50 of 642 μM. In this experiment, it was discovered that a combination of 37 parts of astaxanthin to 1 part of resveratrol provided a statistically and biologically significant increase in the antioxidant efficacy of astaxanthin.

### EXAMPLE 3

### Antioxidant Synergy of Astaxanthin and L-Ergothioneine

This example illustrates the antioxidant effect of combinations of astaxanthin and ergothioneine. The experiment was performed as described in EXAMPLE 1, except that the second compound was ergothioneine, obtained from Sigma (St. Louis, Mo.). Table 4 indicates an EC50 value of 7.77 μM for the combination of astaxanthin with ergothioneine when the components exist in a ratio of approximately 26:1 (astaxanthin:ergothioneine).

TABLE 4
Statistical results of combining astaxanthin with genistein

| Combination | EC50 (μM) | COMBINATION INDEX* |
|---|---|---|
| Astaxanthin: Ergothioneine | 7.77 | 0.864 |
| (26:1) | | |

*Exhabited significant (p <0.5) synergy with CI <1.0.

This combination effectively increased the antioxidant potency of astaxanthin 72-fold; astaxanthin alone exhibited an EC50 of 642 μM. In this experiment, it was discovered that a combination of 26 parts of astaxanthin to 1 part of ergothioneine provided a statistically and biologically significant increase in the antioxidant efficacy of astaxanthin.

### EXAMPLE 4

### Antioxidant Synergy of Astaxanthin and Genistein

This example illustrates the antioxidant effect of combinations of astaxanthin and genistein. The experiment was performed as described in EXAMPLE 1, except that the second compound was genistein, obtained from Sigma (St. Louis, Mo.). Table 5 indicates an EC50 value of 8.89 μM for the combination of astaxanthin with genistein when the components exist in a ratio of approximately 19:1 (astaxanthin:genistein).

TABLE 5
Statistical results of combining astaxanthin with genistein

| Combination | EC50 (μM) | COMBINATION INDEX* |
|---|---|---|
| Astaxanthin: Genistein | 8.89 | 0.665 |
| (19:1) | | |

*Exhibited significant (p <0.5) synergy with CI <1.0.

This combination effectively increased the antioxidant potency of astaxanthin 72-fold; astaxanthin alone exhibited an EC50 of 642 μM. In this experiment, it was discovered that a combination of 19 parts of astaxanthin to 1 part of genistein provided a statistically and biologically significant increase in the antioxidant efficacy of astaxanthin.

### EXAMPLE 5

### Antioxidant Synergy of Astaxanthin and Oleanolic Acid

This example illustrates the antioxidant effect of combinations of astaxanthin and oleanolic acid. The experiment was performed as described in EXAMPLE 1, except that the second compound was oleanolic acid, obtained from Sigma (St. Louis, Mo.). Table 6 indicates an EC50 value of 296 μM for the combination of astaxanthin with oleanolic acid when the components exist in a ratio of approximately 1.6:1 (astaxanthin:oleanolic acid).

TABLE 6
Statistical results of combining astaxanthin with oleanolic acid

| Combination | EC50 (μM) | COMBINATION INDEX* |
|---|---|---|
| Astaxanthin: Oleanolic | 296 | 0.690 |
| Acid (1.6:1) | | |

*Exhibited significant (p <0.5) synergy with CI <1.0.

This combination effectively increased the antioxidant potency of astaxanthin 2.2-fold; astaxanthin alone exhibited an EC50 of 642 μM. In this experiment, it was discovered that 1 to 2 parts of astaxanthin to 1 part of oleanolic acid provided a statistically and biologically significant increase in the antioxidant efficacy of astaxanthin.

### EXAMPLE 6

### Antioxidant Synergy of Astaxanthin and Ascorbic Acid

This example illustrates the antioxidant effect of combinations of astaxanthin and ascorbic acid. The experiment was performed as described in EXAMPLE 1, except that the second compound was ascorbic acid, which was obtained from Sigma (St. Louis, Mo.). Table 7 indicates an EC50 value of 16.3 μM for the combination of astaxanthin with ascorbic acid when the components exist in a ratio of approximately 16:1 (astaxanthin:ascorbic acid).

TABLE 7
Statistical results of combining astaxanthin with ascorbic acid

| Combination | EC50 (μM) | COMBINATION INDEX* |
|---|---|---|
| Astaxanthin: Ascorbic | 16.3 | 0.994 |
| Acid (16:1) | | |

*Exhibited significant (p <0.5) synergy with CI <1.0.

This combination effectively increased the antioxidant potency of astaxanthin 40-fold; astaxanthin alone exhibited an EC50 of 642 μM. In this experiment, it was discovered that only small amounts of ascorbic acid were necessary to provide a large increase in the antioxidant efficacy of astaxanthin.

### EXAMPLE 7

### Antioxidant Synergy of Astaxanthin Containing Algal Meal and the Oil Extractable Components of Ginseng

This example illustrates the antioxidant effect of combinations of astaxanthin and the oil-extractable components of ginseng. The experiment was performed as described in EXAMPLE 1, except that both materials were obtained from retail sources. The astaxanthin sample used was a commercial preparation of Haematococcus pluvialis containing 2 percent astaxanthin and 0.2 percent total of a mixture of zeaxanthin, cantaxanthin, β-carotene and adinorubin. It was obtained from AstaCarotene (Gustavsberg, Sweden). Of the 2 percent astaxanthin in the H. pluvialis extract approximately 80 percent was in the form of monoesters, 15 percent as diesters and the remaining 5 percent as non-esterified astaxanthin. The astaxanthin esters were predominately fatty acid esters in this natural product. For the second product, a glycerol-extract of ginseng was obtained from Nature's Way Products (Springville, Utah). Dose-response curves were described with each test article separately and then in a two-way combination.

Table 8 lists the summary of the testing; an EC₅₀ value of 259 μg/mL was obtained for a 1:9 combination of algal meal and the glycerol-extract of ginseng. The average CI for the dose-response curve was 0.708 indicating strong synergy over the entire range of concentrations.

TABLE 8
Statistical results of combining algal meal with glycerol-extract of ginseng

| | | |
|---|---|---|
| COMBINATION | EC₅₀ (μG/ML) | COMBINATION INDEX* |

| | | |
|---|---|---|
| Algal meal: Ginseng (1:9) | 259 | 0.708 |

*averaged over the EC₅₀, ED₇₅ and EC₉₀ of the dose-response curve; exhibited significant (p <0.5) synergy with CI <1.0.

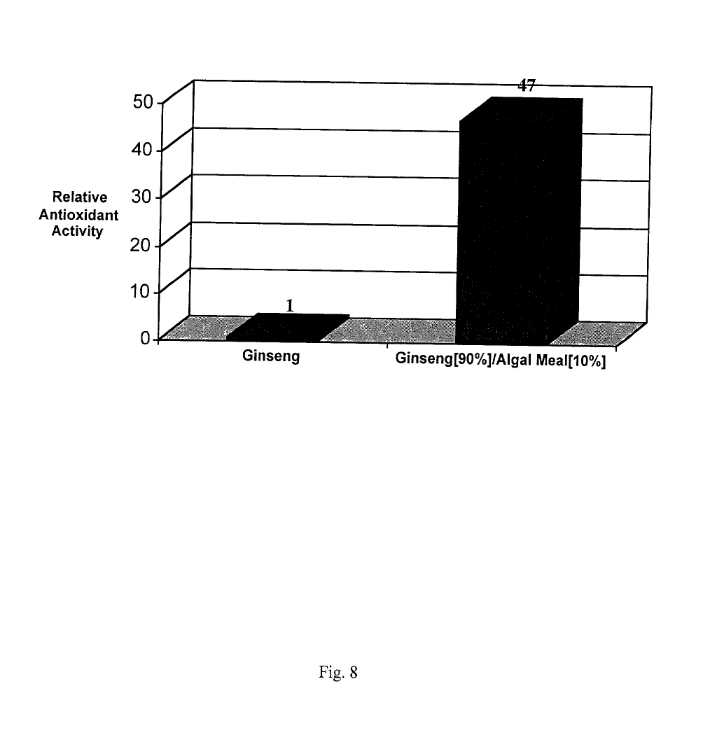

This combination effectively increased the antioxidant potency of the ginseng extract 47-fold (FIG. 8); algal meal alone exhibited an EC50 of 37 μg/mL. In this experiment, it was discovered that only a small amount of algal meal (10%) was necessary to provide a large increase in the antioxidant efficacy of ginseng.

### EXAMPLE 8

### Antioxidant Synergy of Astaxanthin Containing Algal Meal and the Olive Oil Extractable Components of Garlic

This example illustrates the antioxidant effect of combinations of astaxanthin and the olive oil-extractable components of garlic. The experiment was performed as described in EXAMPLE 1, except that both materials were obtained from retail sources. The astaxanthin sample used was a commercial preparation of Haematococcus pluvialis containing 2 percent astaxanthin and 0.2 percent total of a mixture of zeaxanthin, cantaxanthin, β-carotene and adinorubin. It was obtained from AstaCarotene (Gustavsberg, Sweden). Of the 2 percent astaxanthin in the H. pluvialis extract approximately 80 percent was in the form of monoesters, 15 percent as diesters and the remaining 5 percent as non-esterified astaxanthin. The astaxanthin esters were predominately fatty acid esters in this natural product. For the second product, an olive oil-extract of garlic was obtained from Nature's Way Products (Springville, Utah). Dose-response curves were described with each test article separately and then in a two-way combination.

Table 9 lists the summary of the testing; an EC₅₀ value of 483 μg/mL was obtained for a 1:21 combination of algal meal and the olive oil-extract of garlic. The average CI for the dose-response curve was 0.766 indicating strong synergy over the entire range of concentrations.

TABLE 9
Statistical results of combining algal meal with an olive oil-extract of
garlic

| Combination | EC₅₀ (μG/ML) | COMBINATION INDEX* |
|---|---|---|
| Algal meal: Garlic (1:21) | 483 | 0.766 |

*averaged over the EC₅₀, ED₇₅ and EC₉₀ of the dose-response curve; exhibited significant (p <0.5) synergy with CI <1.0.

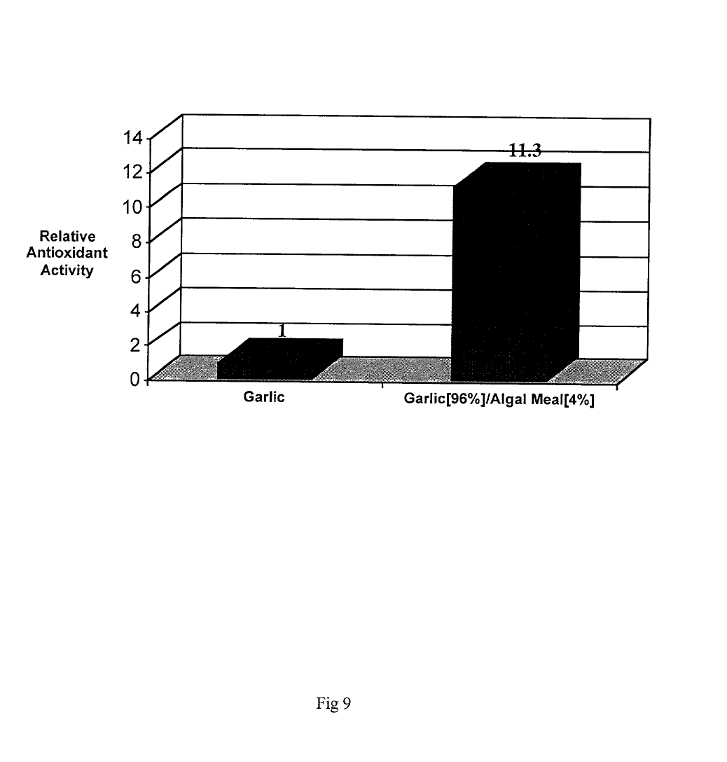

This combination effectively increased the antioxidant potency of the garlic extract 11-fold (FIG. 9); the algal meal alone exhibited an EC50 of 37 μg/mL. In this experiment, it was discovered that only a small amount of algal meal (4%) was necessary to provide a large increase in the antioxidant efficacy of garlic.

### EXAMPLE 9

### Antioxidant Synergy of Astaxanthin Containing Algal Meal and the Alcohol Extractable Components of Ginkgo Biloba

This example illustrates the antioxidant effect of combinations of astaxanthin and the alcohol-extractable components of Ginkgo biloba. The experiment was performed as described in EXAMPLE 1, except that both materials were obtained from retail sources. The astaxanthin sample used was a commercial preparation of Haematococcus pluvialis containing 2 percent astaxanthin and 0.2 percent total of a mixture of zeaxanthin, cantaxanthin, β-carotene and adinorubin. It was obtained from AstaCarotene (Gustavsberg, Sweden). Of the 2 percent astaxanthin in the H. pluvialis extract approximately 80 percent was in the form of monoesters, 15 percent as diesters and the remaining 5 percent as non-esterified astaxanthin. The astaxanthin esters were predominately fatty acid esters in this natural product. For the second product, an olive oil-extract of Ginkgo biloba was obtained from Nature's Answer (Hauppauge, N.Y.). Dose-response curves were described with each test article separately and then in a two-way combination.

Table 10 lists the summary of the testing; an EC₅₀ value of 542 μg/mL was obtained for a 1:18 combination of algal meal and the alcohol-extract of Ginkgo biloba. The average CI for the dose-response curve was 0.975 indicating strong synergy over the entire range of concentrations.

TABLE 10
Statistical results of combining algal meal with an olive oil-extract of
Ginkgo biloba

| Combination | EC₅₀ (μG/ML) | COMBINATION INDEX* |
|---|---|---|
| Algal meal: Ginkgo biloba | 542 | 0.975 |
| (1:18) | | |

*averaged over the EC₅₀, ED₇₅ and EC₉₀ of the dose-response curve; exhibited significant (p <0.5) synergy with CI <1.0.

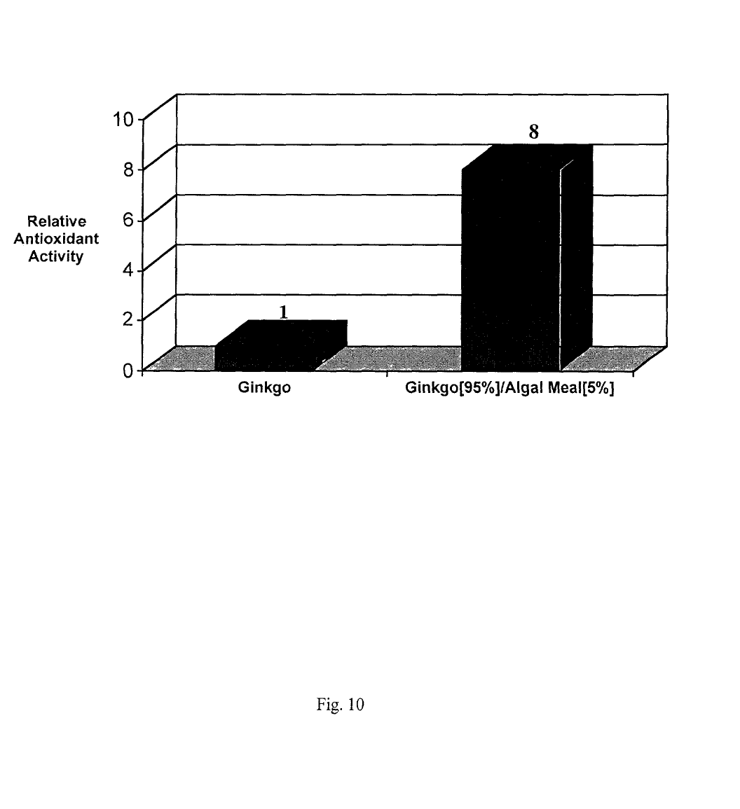

This combination effectively increased the antioxidant potency of the Ginkgo biloba extract 8-fold (FIG. 10); the algal meal alone exhibited an EC50 of 37 μg/mL. In this experiment, it was discovered that only a small amount of algal meal (˜5%) was necessary to provide a large increase in the antioxidant efficacy of Ginkgo biloba.

Thus, among the various formulations taught there has been disclosed a formulation comprising as a first component, a carotenoid species, and, as a second component, at least one member selected from the group consisting of lipoic acid, dihydrolipoic acid (DHLA), a stibene species, ergothioneine, an isoflavone species, a triterpene species, ascorbic acid and derivatives thereof. These combinations provide for a synergistic anti-oxidant activity. It will be readily apparent to those skilled in the art that various changes and modifications of an obvious nature may be made without departing from the spirit of the invention, and all such changes and modifications are considered to fall within the scope of the invention as defined by the appended claims. Such changes and modifications would include, but not be limited to, the incipient ingredients added to affect the capsule, tablet, lotion, food or bar manufacturing process as well as vitamins, herbs, flavorings and carriers. Other such changes or modifications would include the use of other herbs or botanical products containing the combinations of the present invention disclosed above.

We claim:

1. A composition having synergistic antioxidant activity comprising an effective amount a first component of a carotenoid species, and, as a second component, at least one member selected from the group consisting of lipoic acid, dihydrolipoic acid (DHLA), a stilbene species, ergothioneine, a flavone species, a triterpene species, ascorbic acid and derivatives thereof.

2. The composition of claim 1 wherein first and second components are derived from microorganisms, plants, extracts of microorganisms or plants.

3. The composition of claim 1 wherein said first and second components are synthetic compounds.

4. The composition of claim 1 wherein at least one of said first or second components is conjugated with a compound selected from the group consisting of mono- or di- saccharides, amino acids, fatty acids, sulfates, succinate, acetate and glutathione.

5. The composition of claim 1, additionally containing one or more members selected from the group consisting of antioxidants, vitamins, minerals, proteins, fats, carbohydrates, glucosamine, chondrotin sulfate and aminosugars.

6. A composition having synergistic antioxidant activity comprising an effective amount a first component of a carotenoid species selected from the group consisting of astaxanthin, beta-carotene, lutein, lycopene, zeaxanthn and cantaxanthin, and, as a second component, at least one member selected from the group consisting of lipoic acid, dihydrolipoic acid (DHLA), a stilbene species, ergothioneine, a flavone species, a triterpene species, ascorbic acid and derivatives thereof.

7. The composition of claim 6 wherein first and second components are derived from microorganisms, plants, extracts of microorganisms or plants.

8. The composition of claim 6 wherein said first and second components are synthetic compounds.

9. The composition of claim 6 wherein at least one of said first or second components is conjugated with a compound selected from the group consisting of mono- or di- saccharides, amino acids, fatty acids, sulfates, succinate, acetate and glutathione.

10. The composition of claim 6, additionally containing one or more members selected from the group consisting of antioxidants, vitamins, minerals, proteins, fats, carbohydrates, glucosamine, chondrotin sulfate and aminosugars.

11. A composition having synergistic antioxidant activity comprising an effective amount a first component of a pharmaceutical grade carotenoid species selected from the group consisting of astaxanthin, beta-carotene, lutein and lycopene; and, as a second component, at least one member selected from the group consisting of lipoic acid, dihydrolipoic acid (DHLA), a stilbene species, ergothioneine, a flavone species, a triterpene species, and ascorbic acid.

12. The composition of claim 11 wherein first and second components are derived from microorganisms, plants, extracts of microorganisms or plants.

13. The composition of claim 11 wherein said first and second components are synthetic compounds.

14. The composition of claim 11 wherein at least one of said first or second components is conjugated with a compound selected from the group consisting of mono- or di- saccharides, amino acids, fatty acids, sulfates, succinate, acetate and glutathione.

15. The composition of claim 11, additionally containing one or more members selected from the group consisting of antioxidants, vitamins, minerals, proteins, fats, carbohydrates, glucosamine, chondrotin sulfate and aminosugars.

16. A composition having synergistic antioxidant activity comprising an effective amount a first component of a pharmaceutical grade astaxanthin; and, as a second component, at least one member selected from the group consisting of lipoic acid, dihydrolipoic acid (DHLA), a stilbene species, ergothioneine, a flavone species, a triterpene species, and ascorbic acid.

17. The composition of claim 16 wherein first and second components are derived from microorganisms, plants, extracts of microorganisms or plants.

18. The composition of claim 16 wherein said first and second components are synthetic compounds.

19. The composition of claim 16 wherein at least one of said first or second components is conjugated with a compound selected from the group consisting of mono- or di- saccharides, amino acids, fatty acids, sulfates, succinate, acetate and glutathione.

20. The composition of claim 16, additionally containing one or more members selected from the group consisting of antioxidants, vitamins, minerals, proteins, fats, carbohydrates, glucosamine, chondrotin sulfate and aminosugars.

21. A composition having synergistic antioxidant activity comprising an effective amount a first component of a carotenoid species selected from the group consisting of astaxanthin, beta-carotene, lutein, lycopene, zeaxanthn and cantaxanthin, and, as a second component, at least one member selected from the group consisting of lipoic acid, dihydrolipoic acid (DHLA), resveratrol, piceatannol, ergothioneine, genistein, daidzein, glycitein, formonoetin, genisti, daizin, oleanolic acid, ursolic acid, betulin, tripterin, glycyrrhizic acid, and ascorbic acid.

22. The composition of claim 21 wherein first and second components are derived from microorganisms, plants, extracts of microorganisms or plants.

23. The composition of claim 21 wherein said first and second components are synthetic compounds.

24. The composition of claim 21 wherein at least one of said first or second components is conjugated with a compound selected from the group consisting of mono- or di- saccharides, amino acids, fatty acids, sulfates, succinate, acetate and glutathione.

25. The composition of claim 21, additionally containing one or more members selected from the group consisting of antioxidants, vitamins, minerals, proteins, fats, carbohydrates, glucosamine, chondrotin sulfate and aminosugars.

26. A composition having synergistic antioxidant activity comprising an effective amount a first component of a pharmaceutical grade carotenoid species selected from the group consisting of astaxanthin, beta-carotene, lutein and lycopene; and, as a second component, at least one member selected from the group consisting of lipoic acid, dihydrolipoic acid (DHLA), resveratrol, ergothioneine, genistein, daidzein, glycitein, oleanolic acid, ursolic acid, betulin, tripterin, glycyrrhizic acid, and ascorbic acid.

27. The composition of claim 26 wherein first and second components are derived from microorganisms, plants, extracts of microorganisms or plants.

28. The composition of claim 26 wherein said first and second components are synthetic compounds.

29. The composition of claim 26 wherein at least one of said first or second components is conjugated with a compound selected from the group consisting of mono- or di- saccharides, amino acids, fatty acids, sulfates, succinate, acetate and glutathione.

30. The composition of claim 26 additionally containing one or more members selected from the group consisting of antioxidants, vitamins, minerals, proteins, fats, carbohydrates, glucosamine, chondrotin sulfate and aminosugars.

31. A composition having synergistic antioxidant activity comprising an effective amount a first component of a pharmaceutical grade astaxanthin; and, as a second component, at least one pharmaceutical grade compound selected from the group consisting of lipoic acid, dihydrolipoic acid (DHLA), resveratrol, ergothioneine, genistein, daidzein, glycitein, oleanolic acid, ursolic acid, betulin, tripterin, glycyrrhizic acid, and ascorbic acid.

32. The composition of claim 31 wherein first and second components are derived from microorganisms, plants, extracts of microorganisms or plants.

33. The composition of claim 31 wherein said first and second components are synthetic compounds.

34. The composition of claim 31 wherein at least one of said first or second components is conjugated with a compound selected from the group consisting of mono- or di- saccharides, amino acids, fatty acids, sulfates, succinate, acetate and glutathione.

35. The composition of claim 31, additionally containing one or more members selected from the group consisting of antioxidants, vitamins, minerals, proteins, fats, carbohydrates, glucosamine, chondrotin sulfate and aminosugars.

36. A composition having synergistic antioxidant activity comprising an effective amount a first component of a pharmaceutical grade astaxanthin; and, as a second component, at least one pharmaceutical grade compound selected from the group consisting of lipoic acid, dihydrolipoic acid (DHLA), resveratrol, ergothioneine, genistein, oleanolic acid, ursolic acid, and ascorbic acid.

37. The composition of claim 36 wherein first and second components are derived from microorganisms, plants, extracts of microorganisms or plants.

38. The composition of claim 36 wherein said first and second components are synthetic compounds.

39. The composition of claim 36 wherein at least one of said first or second components is conjugated with a compound selected from the group consisting of mono- or di- saccharides, amino acids, fatty acids, sulfates, succinate, acetate and glutathione.

40. The composition of claim 36, additionally containing one or more members selected from the group consisting of antioxidants, vitamins, minerals, proteins, fats, carbohydrates, glucosamine, chondrotin sulfate and aminosugars.

41. A method for normalization or therapeutic treatment of symptoms of oxidative stress in animals comprising administering to an animal a composition comprising effective amount a first component of a carotenoid species, and, as a second component, at least one member selected from the group consisting of lipoic acid, dihydrolipoic acid (DHLA), a stilbene species, ergothioneine, a flavone species, a triterpene species, ascorbic acid and derivatives thereof; and continuing said administration until said symptoms of oxidative stress are reduced.

42. The method of claim 41 wherein the composition is formulated in a dosage form such that said administration provides 1 to 50 mg/day of a cartenoid species, and 10 to 1200 mg/day lipoic acid or dihydrolipoic acid, 1 to 1000 mg/day of a stilbene species, 1 to 50 mg/day of ergothioneine, 0.5 to 500 mg/day of a flavone species, 2 to 1000 mg/day of a triterpene species, or 50 to 10,000 mg/day ascorbic acid.

43. The method of claim 42, wherein the composition is administered in an amount sufficient to maintain a serum or tissue concentration of 0.01 to 5,500 μM of a cartenoid species, and 0.08 to 50 μM lipoic acid or dihydrolipoic acid, 0.005 to 50 μM of a stilbene species, 0.01 to 3,000 μM of ergothioneine, 0.02 to 800 μM of a flavone species, 0.05 to 3,500 μM of a triterpene species, or 0.01 to 500 μM ascorbic acid.

44. The method of claim 42 wherein said animal is selected from the group consisting of humans, non-human primates, dogs, cats, birds, horses and ruminants.

45. The method of claim 42 wherein administration is by a means selected from the group consisting of oral, parenteral, topical, transdermal and transmucosal delivery.

46. The method of claim 45 wherein the topical application formula contains 0.001 to 10 wt % of the first component and 0.001 to 10 wt % of the second component.

47. The method of claim 42 wherein the oxid ative stress symptoms is associated to one or more member selected from the group consisting of cardivasucular disorders, immune system disorders, cataracts and macular degeneration, aging, decreased growth rate, lack of energy, cognitive function disorders and stomach fimction disorders.

48. The method of claim 42 wherein the composition is administered in a form selected from the group consisting of capsules, tablets, lotions, food bars, chewable gums, cereal, diary products, and snacks.

49. The method of claim 42 wherein the first component and the second component are administered in a sequential manner.

50. The method of claim 49 wherein the first component and the second component are administered in a substantially simultaneous manner.

51. A method for normalization or therapeutic treatment of symptoms of oxidative stress in animals comprising administering to an animal a composition having synergistic antioxidant activity comprising an effective amount a first component of a carotenoid species selected from the group consisting of astaxanthin, beta-carotene, lutein, lycopene, zeaxanthn and cantaxanthin, and, as a second component, at least one member selected from the group consisting of lipoic acid, dihydrolipoic acid (DHLA), a stilbene species, ergothioneine, a flavone species, a triterpene species, ascorbic acid and derivatives thereof; and continuing said administration until said symptoms of oxidative stress are reduced.

52. A method for normalization or therapeutic treatment of symptoms of oxidative stress in animals comprising administering to an animal a composition having synergistic antioxidant activity comprising an effective amount a first component of a pharmaceutical grade carotenoid species selected from the group consisting of astaxanthin, beta-carotene, lutein and lycopene; and, as a second component, at least one member selected from the group consisting of lipoic acid, dihydrolipoic acid (DHLA), a stilbene species, ergothioneine, a flavone species, a triterpene species, and ascorbic acid; and continuing said administration until said symptoms of oxidative stress are reduced.

53. A method for normalization or therapeutic treatment of symptoms of oxidative stress in animals comprising administering to an animal a composition having synergistic antioxidant activity comprising an effective amount a first component of a pharmaceutical grade astaxanthin; and, as a second component, at least one member selected from the group consisting of lipoic acid, dihydrolipoic acid (DHLA), a stilbene species, ergothioneine, a flavone species, a triterpene species, and ascorbic acid; and continuing said administration until said symptoms of oxidative stress are reduced.

54. A method for normalization or therapeutic treatment of symptoms of oxidative stress in animals comprising administering to an animal a composition having synergistic antioxidant activity comprising an effective amount a first component of a carotenoid species selected from the group consisting of astaxanthin, beta-carotene, lutein, lycopene, zeaxanthn and cantaxanthin, and, as a second component, at least one member selected from the group consisting of lipoic acid, dihydrolipoic acid (DHLA), resveratrol, piceatannol, ergothioneine, genistein, daidzein, glycitein, formonoetin, genisti, daizin, oleanolic acid, ursolic acid, betulin, tripterin, glycyrrhizic acid, and ascorbic acid; and continuing said administration until said symptoms of oxidative stress are reduced.

55. A method for normalization or therapeutic treatment of symptoms of oxidative stress in animals comprising administering to an animal a composition having synergistic antioxidant activity comprising an effective amount a first component of a pharmaceutical grade carotenoid species selected from the group consisting of astaxanthin, beta-carotene, lutein and lycopene; and, as a second component, at least one member selected from the group consisting of lipoic acid, dihydrolipoic acid (DHLA), resveratrol, ergothioneine, genistein, daidzein, glycitein, oleanolic acid, ursolic acid, betulin, tripterin, glycyrrhizic acid, and ascorbic acid; and continuing said administration until said symptoms of oxidative stress are reduced.

56. A method for normalization or therapeutic treatment of symptoms of oxidative stress in animals comprising administering to an animal a composition having synergistic antioxidant activity comprising an effective amount a first component of a pharmaceutical grade astaxanthin; and, as a second component, at least one pharmaceutical grade compound selected from the group consisting of lipoic acid, dihydrolipoic acid (DHLA), resveratrol, ergothioneine, genistein, oleanolic acid, ursolic acid, and ascorbic acid; and continuing said administration until said symptoms of oxidative stress are reduced.
